(19) United States
(12) Patent Application Publication
(10) Pub. No.: US 2006/0015041 A1
(43) Pub. Date: Jan. 19, 2006

(54) Periodontal pocket depth recorder

(76) Inventors: Brian Neal, Fortuna, CA (US); Mary Ann Norton, (US)

Correspondence Address: US CORPORATIONS, INC., 630 NEPTUNE AVE., ENCINITAS, CA, 92024, US

(21) Appl. No.: 10/890,720

(22) Filed: Jul. 13, 2004

(51) Int. Cl. A61C 19/04; A61C 19/00; A61B 5/103

(52) U.S. Cl. 600/587; 33/513; 433/25

(57) ABSTRACT

A dental depth locating device comprised of an integrated base unit and software, three pedal position foot controls, standard printer, and a re-printable template capable of providing an economical means of performing periodontal depth charting quickly and easily by one person with minimal risk of contamination.

### CROSS-REFERENCE TO RELATED APPLICATION

Not applicable

### STATEMENT REGARDING FEDERALLY SPONSORED RESEARCH OR DEVELOPMENT

Not applicable

### REFERENCE TO SEQUENCE LISTING

Not applicable

### BACKGROUND OF THE INVENTION

This invention relates to a dental depth locator capable of providing an economical means of performing periodontal depth charting quickly and easily by one person with minimal risk of contamination.

It is common practice in the typical dental office to have some manner to record the general periodontal health of the patient. One method that is commonly used is to record the depths of pockets in the gums surrounding each tooth. As the periodontal health of the patient's mouth deteriorates, these pockets will increase in depth. The general practice is to record at 6 locations around each tooth. The mouth has 32 teeth if all are still present, resulting in 196 potential pocket depths that would need to be recorded, a considerable number of measurements.

In current practice one of two methods are commonly used. One way is to have 2 people involved in the recording; one to measure and dictate the measurements, another to write them down. It is often impractical in a busy dental office to have 2 people available to do the recording, as well as expensive. The other method is for one person to make some measurements, typically about 3, and then write them down on a chart on their lap or nearby table. This method is both slow and creates considerable risk of contamination, as the dentist or hygienist must either remove their gloves and then put them on again during each writing session, or attempt to maintain the sterility of the pen and chart with sterile plastic bags, something that is very difficult to do. For these reasons many offices do not do periodontal charting as often as they would like.

In terms of current products designed to address this problem, those we have been able to locate after considerable search fall into only two categories. All require the use of a complete computer system. Some rely on a voice recognition program to input the data. These can be unreliable and difficult to use. Other systems incorporate a measurement tool that actually measures, via mechanical, ultrasonic or temperature methods the pocket depth with the tool itself. These systems also are designed to attach to a P.C. compatible computer and create a very elaborate chart of the entire mouth. There are 2 drawbacks of these systems for the typical dental office. 1) The room required such a system is not available in many dental offices. 2) The cost of these systems ($2,000+ for voice recognition, $20,000+ for those incorporating a measuring probe), is not financially viable for many dentists.

The product we have developed is designed to provide an economical means for a single person to do periodontal depth charting quickly with minimal risk of contamination.

The system can be considered to contain 4 components. A) The main unit is a small, battery operated box containing a micro-controller to accept inputs from the footswitch, a data storage area, an output to a small thermal label printer, control buttons and an LCD display, LED indicators and a speaker as feedback to the operator as to the information that has been stored, next location to store and which footswitch to use for data input. B) The 3 pedal footswitch is used to select the location to store and to store the data. C) The thermal printer is used to print out the stored measurements onto a small label once the recording session is done. D) The template provides a full mouth chart and locations to apply labels so the measurements can be easily read.

While the first product we will be producing will incorporate a footswitch for data input and a printer to output the data, we wish to include in the patent application that the footswitch can be replaced with a microphone for voice recognition as the input, as well as the printer replaced by a connection to a PC so that the data could be input into a software program of our creation and stored on the PC or output to a standard printer.

The driver for one particular brand and type of thermal printer are incorporated in the main unit. The footswitch and printer are easily detached from the main unit by use of cables and connectors.

### SUMMARY OF THE INVENTION

From the patent search performed and from our own searches on the USPTO website, we can find only one patent that closely approximates our product. It is the product described in U.S. Pat. No. 4,665,621. This is a very old (May 19, 1987) and very extensive patent, covering all aspects of the device and operation. Several aspects of this patent closely correspond to our product. I have attempted to list below the similarities and differences between the product described in the patent and ours.

In the following, the term “the product” refers to the electronic assembly described in U.S. Pat. No. 4,665,621.

The underlined statements are the similarities.
1) The product is self-contained. It does require an AC power cord while ours is battery operated.
2) The product is designed to be connected to a printer. The printer described is a full sheet printer, not a small label printer as in our unit. There appears to be no data storage capability in the product, meaning the printer must be continually attached, ours can be connected at the completion of the storage process.
3) The product uses an LCD display showing pocket location and depth stored. It does not incorporate auditory feedback from a speaker or LED indicators as prompts.
4) It product includes a 3 pedal footswitch. The footswitch has a totally different function than on our unit, and is not used to store data.
5) The product uses a storage pattern of tooth and pocket that closely approximates one of the storage pattern options on our unit. Our product has 2 additional storage modes. This pattern is the probably the most commonly used among dentists and dental hygienists as it a logical method to move quickly from tooth to tooth.
6) The most considerable difference between the product and ours is that it incorporates an electronic measuring probe to record the periodontal pocket depth. It may be considered a measuring and recording device. With our unit, the operator uses whatever manual probing tool they like to measure the pocket depth, and only records the data with our device.

### BRIEF DESCRIPTION

The current device is primarily designed as a tool for the dental profession. It is designed to aid in the storing and recording of the depth of the pockets around each tooth in the mouth. We consider the end product to have four major components: A standard off-the-shelf printer (a specific brand and model), a slightly customized off-the-shelf 3-pedal foot-pedal assembly, our electronics assembly (called the base unit) and a re-printable 8.5×11 template to locate and store the labels produced from the printer.

Thus, it is an object of the invention to provide an improved dental depth locating device construction.

A further object of the invention is to provide a dental depth locating device construction which is rugged, economical, easily adjusted to selectable pocket depth recording patterns and to various combinations of tools and implements including but not limited to those discussed.

Another object of the invention is to provide an improved dental depth locating device construction which includes a three pedal foot assembly and related supports designed to improve data storage accuracy and flexibility of the design in the office environment.

Accordingly, it is an object of the present invention to provide a dental depth locating device which embodies an electronic base unit.

Further, it is an object of the present invention to measure pocket depths around each tooth.

Further still, it is an object to provide such a dental depth locating device which will function with as little as one operator.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new dental depth locating device apparatus and which has many of the advantages of the devices mentioned heretofore and many novel features that result in an dental depth locating device which is not anticipated, rendered obvious, suggested, or even implied by any of the prior art tool guides, either alone or in any combination thereof.

It is another object of the present invention to provide a new dental depth locating device which may be easily and efficiently manufactured and marketed.

It is a further object of the present invention to provide a new dental depth locating device which is of a durable and reliable construction.

An even further object of the present invention is to provide a new dental depth locating

### ILLLUSTRATIONS

A perspective view of the present invention as viewed herein;

The Dental Depth Locator and its related parts are herein composed of:
1. Main unit
2. Input Device
3. Microphone/Headphone
4. Thermal Label Printer
5. Personal P.C.

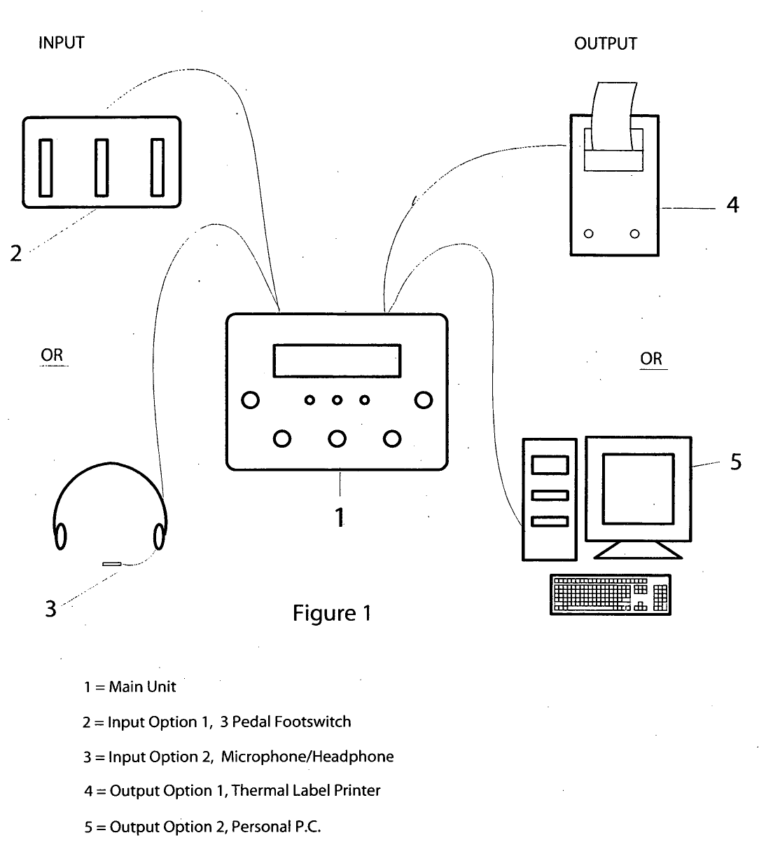

FIG. 1, The input devices described as Pedal Foot switch, Microphone/Headphone, Main Unit, and the output devices; Thermal Paper Printer, Personal PC

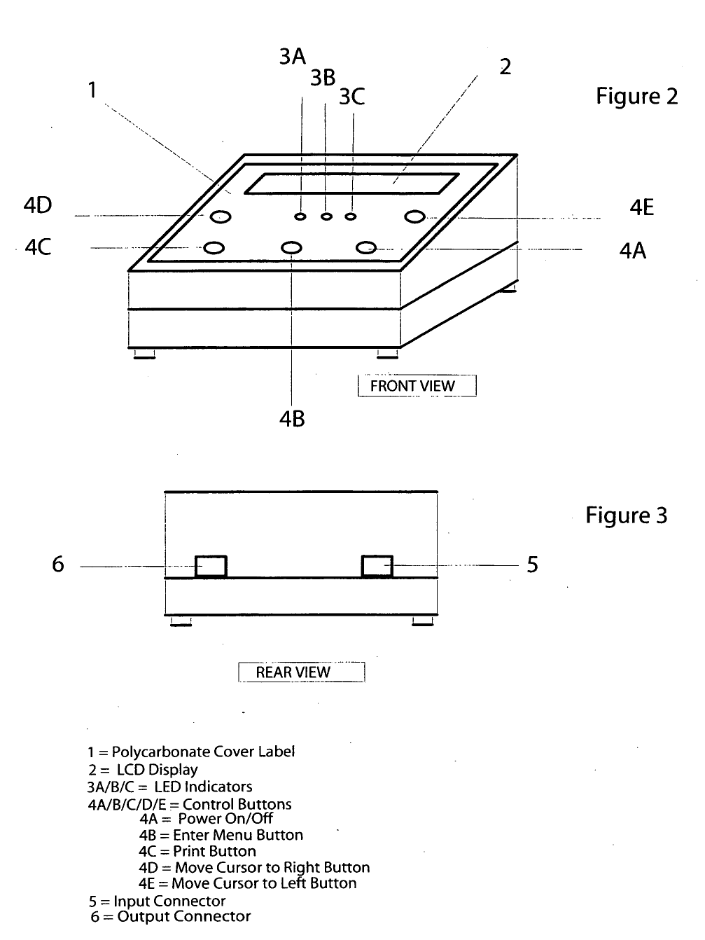

FIG. 2, a perspective view of the Main Unit, front and rear.

Described as; now referring to FIG. 1, a Dental Depth Locator comprised of an input device, FIG. 1 number 2, a pedal foot switch, or number 3 a microphone/headphone which sends information to number 1, the main unit. The main unit, number 1, sends data to one of two receiving devices; number 4 a thermal printer and/or number 5 a personal printer.

The main unit illustrated in FIG. 2, is composed of number 1, a polycarbonate cover label, number 2, a LCD display, number 3A-C LED indicators, number 4A-E, control buttons, number 5, input connectors and number 6, the output connector.

We claim:

1. A dental depth locator capable of providing an economical means of performing periodontal depth charting quickly and easily by one person with minimal risk of contamination.

2. A dental depth locator according to claim 1 wherein the device is battery operated and complete self-contained, does not require an AC plug or computer system, making it very portable.

3. A dental depth locator according to claim 1 wherein the device offers 3 modes of storing the data: the entire mouth, by the 4 mouth quadrants and by selecting individual teeth.

4. A dental depth locator according to claim 1 wherein the device incorporates the printing of just a small label instead of an entire chart. The printer is very small, meaning the entire unit will take very little space in the dental laboratory.

5. A dental depth locator according to claim 1 wherein the device uses flashing LEDS as prompts as to which footswitch assembly pedal(s) the unit is expecting input from.

6. A dental depth locator according to claim 1 wherein the device provides 3 methods of feedback: auditory through the speaker and visual by the LCD display and LEDs.

7. A dental depth locator according to claim 1 wherein the device tooth selection and data storage is accomplished completely through the footswitch assembly.

8. A dental depth locator according to claim 1 wherein the device components on the top of the main unit are sealed by an alcohol resistant plastic label so the unit can be easily wiped clean to disinfect, should they need to touch the main unit

9. A dental depth locator according to claim 1 wherein the device software is designed to minimize the time necessary to record data, automatically incrementing or decrementing the tooth number. Teeth, stored as missing on the facial side, are automatically incremented or decremented past on the lingual side.

10. A dental depth locator according to claim 1 wherein the device software allows for the operator to switch between the three recording modes, for example, to record the pocket depth for the entire top half of the mouth and selected teeth on the bottom, further enhancing the speed in which a desired charting can be accomplished.
